(19) United States
(12) Patent Application Publication
(10) Pub. No.: US 2013/0023910 A1
(43) Pub. Date: Jan. 24, 2013

(54) TISSUE-IDENTIFYING SURGICAL INSTRUMENT

(71) Applicants: CLIFFORD T. SOLOMON, Severna Park, MD (US); THEODORE C. SOLOMON, Hampstead, MD (US)

(72) Inventors: CLIFFORD T. SOLOMON, Severna Park, MD (US); THEODORE C. SOLOMON, Hampstead, MD (US)

(21) Appl. No.: 13/556,121

(22) Filed: Jul. 23, 2012

Related U.S. Application Data
(60) Provisional application No. 61/510,408, filed on Jul. 21, 2011.

(51) Int. Cl. A61B 17/32; A61B 17/08; A61B 17/28

(52) U.S. Cl. 606/158; 606/170; 606/205

(57) ABSTRACT

The tissue-identifying surgical instrument includes a surgical instrument having a handle and an integral probe operatively connected to the handle. The probe senses a tissue of interest to identify the type of tissue, e.g., nerve, muscle, vein or other. The interior of the handle includes a control assembly connected to a power source for operation of the tissue identification function. The control assembly displays and wirelessly transmits tissue identification data to a monitoring workstation to inform the surgeon of the type of tissue contacted by the probe.

### CROSS-REFERENCE TO RELATED APPLICATION

This application claims the benefit of U.S. Provisional Patent Application Ser. No. 61/510,408, filed Jul. 21, 2011.

### FIELD OF THE INVENTION

The present invention relates to surgical instruments, and particularly to a tissue-identifying surgical instrument that includes a tissue-identifying component with integrated multiple functions for more efficient, accurate and safer execution of a surgical procedure.

### DESCRIPTION OF THE RELATED ART

When performing any type of sensitive and delicate surgical procedures such as skull base surgery, neck dissections, carotid endarterectomy, and the like, the surgeon, with the help of a team of technicians, must determine the nature of the tissue under observation to avoid unintended damage to sensitive tissues such as nerves. Typically, the surgeon employs an electrically powered probe wired to a power source and monitoring device to electrically stimulate the tissue of interest in order to determine whether the tissue is a nerve, blood vessel, muscle or other type of tissue. This helps the surgeon map the anatomical section so that any incisions or resections may be performed with some degree of confidence that the procedure will not result in unintended and irreparable harm resulting in functional impairment. For the patient's well-being and ultimate recovery it is imperative to preserve the integrity of nerve tissues as well as vascular support for the tissues as much as possible in these delicate surgical procedures. Any damage to nerve tissue or compromise of blood flow may prolong recovery and/or cause further complications detrimental to the patient's health.

A constant threat to the safety of sensitive tissues in the surgery field is the tedious and time consuming process involved in the dissection of tissues. To avoid unintentionally damaging delicate tissues, the surgeon conventionally employs the use of a probe to identify different tissues in the surgery field before commencing or continuing the process of tissue dissection. The surgeon may hold the probe in one hand while the other hand is occupied with another surgical instrument such as a scalpel. It is also typical that the surgeon grasping the probe will attempt to determine the type of tissue and upon making a determination will set aside the probe, take up the surgical scalpel, and proceed with the tissue dissection. As the surgery progresses, the constant back and forth between the probe and the other surgical instruments delays the surgery and increases the risk of unintended damage to the tissues, thus negatively affecting the safety of the patient. The longer the surgical procedure lasts, the more likely a mistake will be made by the surgeon due to fatigue or lapse in focus. Moreover, a lengthy surgery greatly increases the expense of the procedure and may result in a lengthy recovery time for the patient, which also adds to the resulting increased medical expense.

The requirement for the surgeon to repeatedly manipulate the cumbersome probe and its cable connections during the surgical procedure also has the potential of creating problems or difficulties for the surgeon in his efforts to focus on the surgical procedure without distractions. The typical probe is connected by a probe cable to a monitor, which displays the information obtained by the probe to a member of the surgical team who verbally communicates observations to the surgeon manipulating the probe to tissue of interest within the surgical site. The surgeon's complete dependence on the other man in the loop inherently creates delays between the time the probe contacts the tissue of interest and the time when the surgeon knows what type of tissue he has contacted. Some probes are also operated by the surgeon using external actuators, such as foot pedals. During the surgical procedure, the surgeon is often distracted from his primary purposes by his need to be cognizant of the location of the probe cable and to adjust the position of the probe cable to avoid its interference or entanglement with the surgeon's manipulation of other surgical instruments. In addition, any handling or effort to relocate the probe cable to a safer or less interfering location during the surgical procedure serves to unnecessarily increase the time spent in surgery as well as increase the chances of accidental damage to the tissue in the surgery field due to the surgeon's attention being diverted thereby. Repeated attempts to move the probe cable may lead to aggravation, which negatively impacts the surgeon's focus, and extensive operation of the foot pedal may cause unnecessary fatigue or accidental misplacement. Moreover, the cables associated with the foot pedal can result in the same potential problems as the probe cable.

It would greatly facilitate the work of the surgeon and provide a much higher level of patient safety to provide a method and/or device that would eliminate the problems attendant to the use of conventional tissue identification probes as described above. In addition to reducing distractions and improving conditions for the surgeon, the device by eliminating the problems discussed above could also substantially decrease the time spent in surgery and significantly increase the safety of the surgical procedure for the patient. Thus, a tissue-identifying surgical instrument solving the aforementioned problems is desired

### SUMMARY OF THE INVENTION

The tissue-identifying surgical instrument includes a surgical instrument having a proximal base or handle, a distal operative end, and a tissue identification probe integrated with or adjacent to the distal operative end of the instrument. The probe is provided with at least one and preferably multiple stimulating and/or sensing elements that can be selectively employed by the surgeon to identify the type of tissue, e.g., nerve, muscle, vein or other tissue that has been stimulated or touched by the probe. The base or handle of the instrument includes a probe control assembly connected to a power source. The power source for the instrument is preferably located entirely within the base or handle and more preferably is a rechargeable power cell or battery capable of providing prolonged use. The control assembly is capable of wireless transmission of data collected through its tissue sensing function to a data collection, interpretation, and recording workstation from where tissue identification information can be monitored by a member of the surgical team and simultaneously provided to the surgeon by wirelessly communication. Tissue identification information can also be directly provided to the surgeon through visual indicators that are provided on the handle as well as by wireless transmissions to a heads-up display (HUD) provided for the surgeon on a framework configured to be worn by the surgeon; that framework being similar to surgical loupes, surgical headlights, eye glasses, surgical head bands, and the like. Electrical power sources employed in the components and elements of the invention are preferably rechargeable and more preferably rechargeable by magnetic induction means. Sensor data collection can be immediately provided to the surgeon and medical team as well as saved as a time-line data collection for later recall and analysis if necessary.

These and other features of the present invention will become readily apparent upon further review of the following specification and drawings.

### BRIEF DESCRIPTION OF THE DRAWINGS

FIG. 1 is an environmental, partially transparent, perspective view of a first exemplary embodiment of a tissue-identifying surgical instrument according to the present invention, in the form of a scalpel wherein the tissue-identifying probe is attached to the handle.

FIG. 2 is a schematic diagram of the control assembly for the tissue-identifying surgical instrument of FIG. 1.

FIG. 3 is a partially transparent, perspective view of an alternative embodiment of a tissue-identifying surgical instrument according to the present invention, in the form of a scalpel having multiple probe types.

FIG. 4 is a perspective view of the distal end of the instrument of FIG. 3, wherein the tissue-identifying probes and the probe housing are attached to the detachable scalpel blade and the functional connections of the blade-mounted probes are provided as a quick connect-disconnect to the components in the handle.

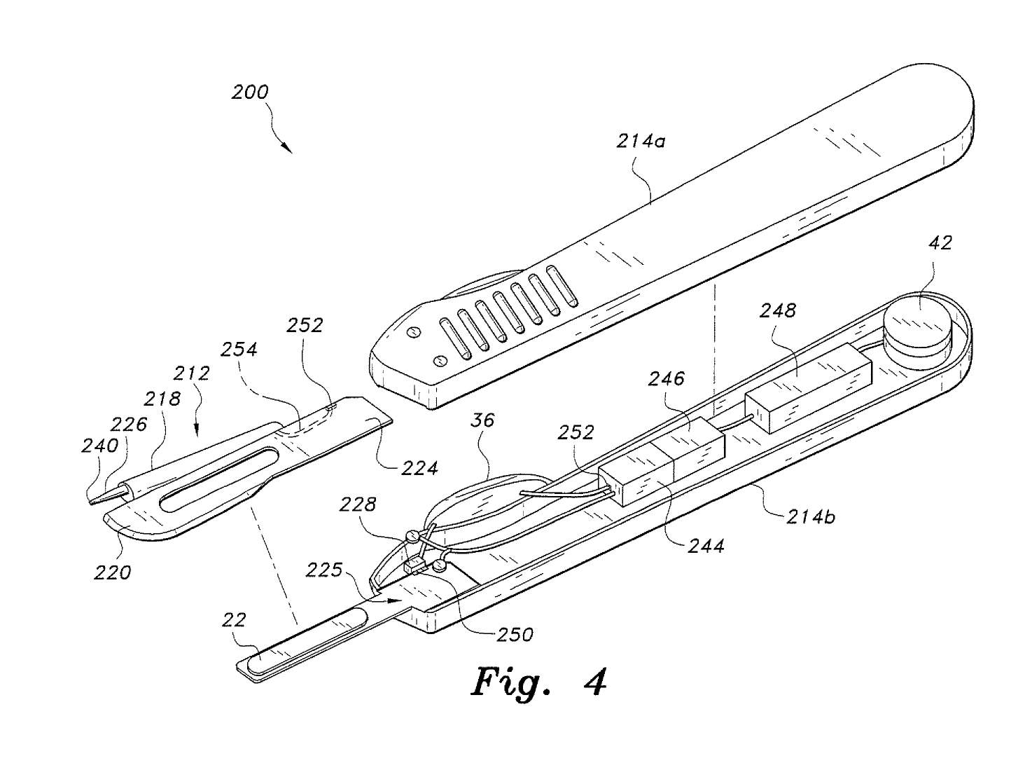

FIG. 5 is a perspective view of another alternative embodiment of a tissue-identifying surgical instrument according to the present invention, in the form of a forceps.

FIG. 6 is a perspective view of another alternative embodiment of a tissue-identifying surgical instrument according to the present invention, in the form of a hemostat.

Similar reference characters denote corresponding features consistently throughout the attached drawings.

### DETAILED DESCRIPTION OF THE PREFERRED EMBODIMENTS

The tissue-identifying surgical instrument, generally shown at 10, includes an integrated tissue identification probe, generally shown at 12, with wireless transmission and/or reception capabilities to improve and expedite tissue identification during surgery and thereby substantially reduce surgery time and many potential hazards to the patient during the operation. In a surgical procedure a dissection can require a plurality of different surgical instruments, many of which could be more beneficial to the surgeon if provided with the additional capability of quick and reliable tissue identification as described herein. Provided herein are descriptions of various examples of common surgical instruments that can include, in addition to their primary function, the additional capability of assisting the surgeon in the identification of tissue types encountered during the surgical procedure. The non-limiting examples of these multifunctional surgical instruments 10 described below are discussed in detail with the understanding that the teachings thereof can apply to virtually all surgical instruments as required. The following non-limiting descriptions are directed toward hand manipulated tissue-identifying surgical instruments 10; however, the base or handle 14 of the surgical instrument 10 can be easily adapted for operable connection to a robotic surgical system.

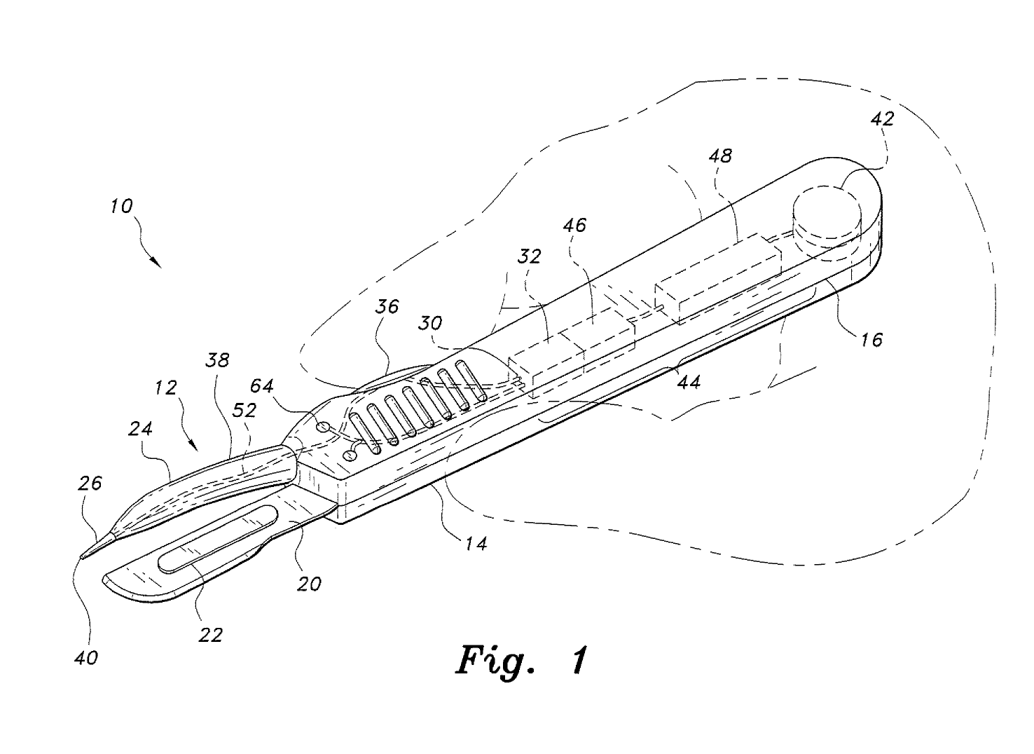

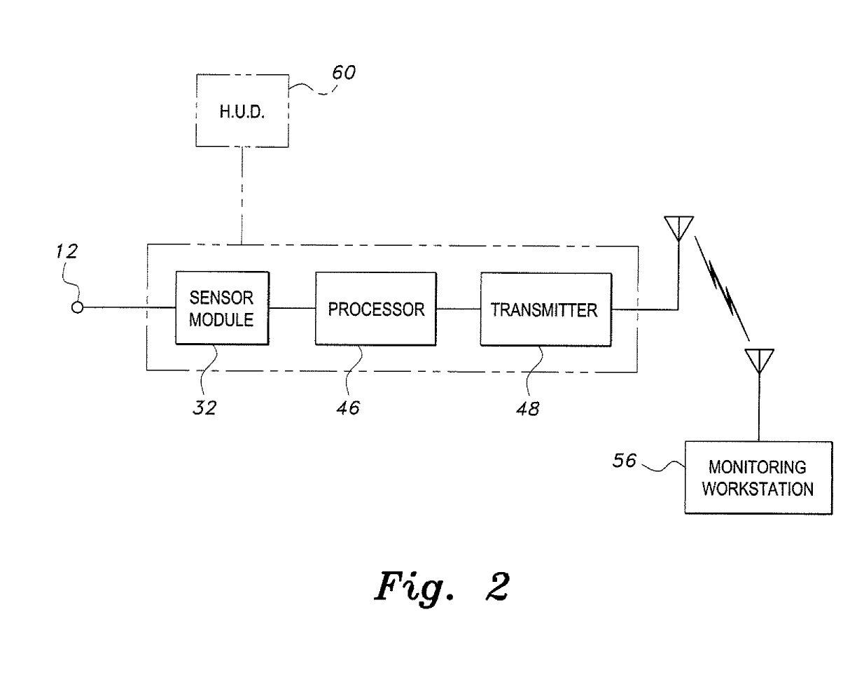

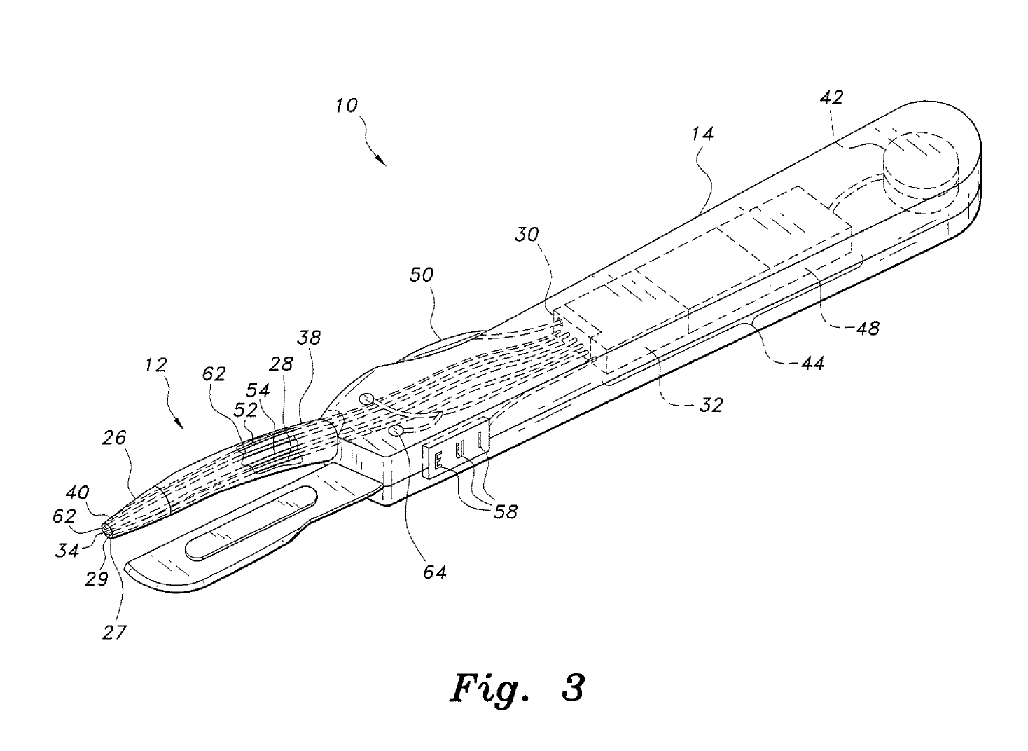

In the exemplary embodiment shown in FIGS. 1 and 2, the tissue-identifying surgical instrument 10 is a scalpel having a base or handle 14 with a proximal gripping end 16 and a distal operable end 18. Also included is a scalpel blade 20, which is removably attached to the distal operable end 18 of the handle 14 by a blade-handle interface 22. The handle 14 of the embodiment shown in FIGS. 1 and 2 includes a probe housing 24 with a probe tip 26 extending proximally from the probe housing 24. The probe housing 24 can be integrally formed with the handle 14 or, alternatively, it can be separately manufactured and attached to the handle 14 during assembly of the instrument 10. The probe tip 26, as shown in FIG. 1, is preferably disposed above the back and away from the scalpel blade 20 so as to provide ease of contact of the probe tip 26 to any tissues of interest while providing clearance for the surgeon to make surgical incisions into the tissue of a patient with minimal or no interference from the probe tip 26. The probe tip 26 can function to act as an electrical probe 40, conducting mild electrical impulses to stimulate the tissue of interest. Examples of such electric probe are taught in U.S. Patent Publication No. 201010317956, published Dec. 16, 2010, and U.S. Pat. No. 7,878,981, issued to Strother et al., the complete disclosures of each being hereby fully incorporated by reference. The electrical impulse from the probe tip 26 elicits a measurable and characteristic response from the tissue of interest, which serves to facilitate the identification of the tissue type, i.e., nerve, blood vessel, muscle or other tissue. As shown in FIG. 3, various other types of probes such as, for example, light probes 29 using visible or invisible light spectra and employing fiber optic carriers to transit from a probe interface 30 incorporated within or attached to a sensor module 32 within the handle 14 to the probe tip 26 can also be provided as single or multiple probe type embodiments of the instrument 10. Other probe types such as radio frequency (RF) 62 and acoustic (ultrasound transmitting/receiving probes and passive listening) probes 34 can also be included with the instrument 10 in the same manner or in conjunction with the electrical and light source probes. Passive listening probes 34 can be employed with the instrument 10 to amplify detected pulsed (arterial) or non-pulsed (venous) flow of blood through arteries and veins that are contacted by the probe tip 26 of the instrument 10. Thus, the instrument 10 can be provided as a probe employing only a single probe, as depicted in FIG. 1 or alternatively, the instrument 10 can be provided with multiple types of probes as described herein or as shown in FIG. 3 wherein the user can, while using the instrument, select the type of probe desired using the selector/actuator 36 provided on the handle 14 and immediately begin using the probe type. Details of non-limiting embodiments of the various types of probes will be discussed below.

A first exemplary embodiment of the instrument 10 is shown in FIG. 1 as a scalpel. As with other commonly used surgical dissectors of this type, the scalpel can be provided with a disposable blade 20 that can be easily attached or detached from the distal end 18 of the handle 14. The probe 12 includes a probe housing 24 that is connected to the distal end 18 of the handle 14 in an overreaching manner above the back edge of the blade 20; however, other orientations of the probe housing 24 to the blade 20 can be used as long as the probe housing 24 and probe tip 26 are advantageously positioned to contact the tissue of interest without interfering with the cutting utility of the scalpel blade 20. The probe housing 24 is preferably constructed from molded, medical grade plastic with a high level of durability required for extended use and repeated sterilization processes. However, any non-conductive or insulating material that protects the surgeon or user from inadvertent electrical shock from operation of the electrical probe 40 and is sufficiently strong and capable of maintaining its structural characteristics through standard usage and sterilization procedures can be used to manufacture the probe housing 24. Examples of suitable materials that can be used for the probe housing 24 include, for example, various elastomers, insulation covered surgical steel, composite, ceramic, and like materials having similar or suitable characteristics. If electrically conductive materials, such as steel, aluminum and the like are used to manufacture the housing 24 a coating or insulating layer 38 over the surface of the housing 24 may be required to protect the user from inadvertent electrical shocks. Unlike the housing 24, the probe tip 26 is preferably manufactured at least partially of an electrical conducting material to facilitate the passage of an electrical charge from the electrical probe 40 to the tissues of interest. The electrical probe 40 can be provided as the only probe provided with the instrument 10, as shown in FIG. 1 or as one of multiple probes as shown in FIG. 3.

A power source 42 can be provided as a detachable battery pack; however it is preferred that the power source 42 is provided as located entirely within the base or handle 14. Preferably the internally located power source 42 is provided as a rechargeable power cell or battery capable of providing sufficient power to support prolonged use of the instrument 10. Most preferable, the power source 42 is sealed within the handle 14 and is rechargeable by magnetic induction means to ensure the moisture proof environment of the interior of the handle 14. In addition to powering the data collection, interpretation, data display, and data transmission operations of the instrument 10, the power source 42 is required to energize the electrical probe 40 to accomplish its tissue stimulating and response sensing functions for the tissue of interest. To maximize electrical conduction through the probe tip 26 to the tissue it is preferred that a highly conductive material such as gold, silver or the like be used on at least a portion of the probe tip 26 although any electrically conductive material would be suitable for manufacture of the probe tip 26 and operation of the electrical probe 40.

The handle 14 provides the structural foundation for the surgical instrument 10 and also provides a probe housing 24 for the components needed to operating the probe, such as the insulated, moisture proof housing 24, the probe tip 26, the electrical probe 40 and other types of probes, such as for example optical or light probes 29, radio frequency 62, and acoustic (passive acoustic, or ultrasound) probes 34. As shown in FIGS. 1 and 2, the handle 14 provides a housing for a control assembly, generally shown at 44. The control assembly 44 includes a sensor module 32, a processor 46, and a wireless transmitter 48, all of which are connected to the power source 42. The sensor module 32 may include a single sensor or probe 12, such as for example an electrical probe 40 as shown in FIG. 1 or it may include multiple probes, non-limiting examples of which are mentioned above and shown in FIG. 3 that can be selected as needed by the surgeon for a specific requirement. While it is preferable that a multiprobe surgical instrument 10, as shown in FIG. 3 includes all necessary components within the sensor module 32 and immediately available and operable as selected by the surgeon, it is also possible that the instrument 10 can be provided with single or selected multiprobe modules, which can be interchangeable or replaceable components that can be switched out as needed with each component having a preselected sensing or probe type.

In the first exemplary embodiment of FIGS. 1 and 2, the sensor module 32 responds to the actuation of the actuator 36 by providing an electrical stimulus from the power source 42 through the electrical probe cable 52 transiting the probe housing 24 to the electrically conductive portion of the probe tip 26. Responsive to the electrical stimulus, the tissue of interest provides a feedback impulse characteristic for the type of tissue stimulated. This response is provided as sensed data that is immediately relayed to the sensor module 32.

The processor 46, a component of the sensor module 32 is operatively connected to the sensor module 32 and serves to convert the sensed data into a transmittable form, which is subsequently passed on to the transmitter 48. The transmitter 48 is capable of wireless communication and can send the processed data simultaneously to multiple possible receivers. The data is wirelessly transmitted to a monitoring workstation 56, which is under close observation by a surgical team member or technician who normally stands by to alert the surgeon of the results. The tissue identification data can also be simultaneously displayed on the tissue type display 58 provided on the handle 14 of the instrument 10 to immediately and automatically inform the surgeon of the tissue type currently being probed. As shown in FIG. 2 the same information can also be simultaneously transmitted to a receiver worn by the surgeon and displayed for the surgeon through a heads-up-display (HUD) 60 provided on a framework configured to be worn by the surgeon; that framework being similar to surgical loupes, surgical headlights, eye glasses, surgical head bands, and the like. U.S. Pat. No. 7,601,119, issued to Shahinian, the complete disclosure of which is fully incorporated herein by reference, discloses the use of HUD technology in robotic surgical procedures.

The wireless transmission of data can be facilitated by a Bluetooth™ type transmission or any other radio frequency transmissions. A Bluetooth™ type transmission system is preferred due to the limited, localized range, security, and the range of digitized data that can be transmitted thereby. The security aspect of the wireless features can include a unique signature for that instrument as a means of identifying the instrument for monitoring purposes. Most important, that unique signal signature can be used to lock into a channel on any medical team receiving station so that subsequently used instruments will not be confused with any other instrument and thereby protect against receiving errant signals from other transmitting devices nearby. The transmitter 48 may also function as a receiver whereby the results from the technician monitoring workstation 56 can be relayed back to the tissue type display 58 on the handle 14 and to the HUD 60 worn by the surgeon as an assurance of the accuracy of data sent to those additional tissue type display sites through other data transfer channels. The provision of multiple channels of data transmission to the surgeon provides redundancy to ensure that the selected probe 12 and the tissue type information provided to the surgeon is correct prior to the surgeon deciding to proceed with the tissue dissection. The tissue identification function of the surgical instrument 10 is rapid, accurate, and safe due to the integrated tissue probe, control assembly, data transmission capabilities, and redundant indicators provided for the surgeon and surgical team.

The prior art tissue identification and dissection procedures practiced by surgeons involves setting aside the surgical instrument in order to use a separate probe device that is hard wired to a remote technician monitored workstation. The surgeon must then wait for the technician to consider the data and verbally report the type of tissue that the technician believes was stimulated by the surgeon. The surgeon then must set aside the cumbersome wired-probe and again take up the first surgical instrument before he can begin to dissect the tissue of interest. In contrast, using the surgical instrument 10 with an integrated wireless tissue-identifying probe, the surgeon, with little hesitation after using that instrument 10 to probe the tissue of interest, can confidently continue the surgical procedure knowing the type of tissue the surgical instrument is touching. The distinct superiority in safety, accuracy, and time management of the surgical procedure is more than beneficial to the surgeon and the patient.

The control assembly 44 includes a manually operated probe selector/actuator 36 that, as shown in FIGS. 1 and 3, can be an actuator button and more preferably an actuator button covered with a moisture proof flexible material. Preferably this moisture proof flexible covering is continuous with the outer surface of the handle 14 so as to provide moisture protection for the entire handle area thus keeping moisture, blood, water and the like from the contaminating the actuator 36 and the control assembly 44. This actuator 36 is functionally connected to the power circuit provided to the sensor module 32 and upon selective manipulation by the surgeon provides the necessary impulse to change modes, that is select a specific type of probe, or to power the selected probe and cause a tissue identifying probe impulse to be emitted from the probe tip to the tissue of interest. As shown in the drawings, the actuator 36 may be disposed on the top spine of the scalpel at a location easily reached by the index finger of the surgeon. The actuator 36 may alternatively be placed at other ergonomically suitable locations for comfort. In the preferred embodiment, the actuator 36 is integral with handle 14 and deformable. Some examples of such an actuator include a button such as a plastic or elastomeric protrusion that deforms when depressed and springs back to original shape upon release, or a mechanical switch covered by plastic or elastomer. Such protected switches have been referred to as “blister buttons” due to their appearance and waterproof characteristics. With such a construction, the scalpel may be easily cleaned, sterilized and reused with minimal to no potential contamination or damage to the electronic or movable parts of the scalpel.

The handle 14 may include a plurality of operating indicator lights 64 for visual confirmation of various functions of the scalpel. A non-limiting, preferred example of operating indicator lights 64 that can be used for the instrument 10 are Light Emitting Diode (LED) indicator lights 64. As a non-limiting example of the possible use for such indicator operating lights 64, one or both of the indicator lights 64 may turn yellow upon a single depression of the actuator 36 to indicate that the probe is powered on and ready for identifying the tissue. For multi-function sensing, the actuator 36 may be depressed repeatedly to cycle through the various sensing functions, that is the types of probes available. In response, one of the indicator lights 64 may turn blue for electric, red for IR, or green for laser, It should be understood that other color code and button-press combinations can be used in addition to the examples discussed herein. The lights 64 may be flush with the outer surface of the handle 14 or be disposed underneath a protective, translucent covering on the handle 14. In the latter case, the translucency permits the colored light to shine through with minimal degradation of the intensity, vibrancy and color of the LED. As an alternative, the indicator lights 64 may also be used to indicate the type of tissue identified by the probe with a color code being assigned to indicate different types of tissue sensed. Preferably, the instrument 10 can be provided with a separate easily observed bank of tissue type identification lights 94 so as not to confuse the meaning of the operating indicator lights 64 with the tissue type identification lights 94.

As briefly discussed earlier, the power source 42 is preferably a rechargeable and reusable battery to minimize environmental impact and permit the power source 42 to be included within the moisture proof handle 14. Non-limiting examples of acceptable battery types include lead-acid, nickel cadmium (NiCd), nickel metal hydride (NTMI-I), lithium ion (Li-ion), and lithium ion polymer (Li-ion polymer) batteries. The long operating life of currently produced batteries assure their successful operation for the duration of even the longest of surgeries; however, as a precaution, if necessary during the course of a surgical procedure, replaceable multiple batteries can be available to be switched out when the initial battery expends its charge. In the event that a single charge for the operating power source, i.e., the battery, is insufficient for the duration of a surgical procedure, it is preferred as more expeditious and reliable to begin the surgical procedure with multiple fully charged scalpels, some of which may be positioned on a battery recharging device and available to be substituted for instruments 10 that have expended their available power prior to the conclusion of the surgical procedure. The foregoing contingencies for prolonged surgeries are only precautionary and not considered necessary for normal operation of the instrument 10.

As mentioned previously, the selected probe may include other sensing and/or identifying functions. As a non-limiting example, the probe type can be an infrared emitter of the type taught in U.S. Pat. No. 6,285,902, issued to Kienzle III et al., which is hereby incorporated in its entirety. Such a probe can be used to image and thereby map the target anatomical area. Another example of a probe type that can be used in the instrument 10 to facilitate the identification of tissue types is a laser probe as taught in U.S. Patent Publication No. 2005/0099824, published May 12, 2005, which is hereby incorporated in its entirety. This type of probe can also be used to illuminate as well as map the tissue of interest. Other types of tissue probes can also be easily adapted for use in the instrument 10. As shown in FIG. 3, similar to the example of the electrical probe earlier described and shown in FIG. 1 having an electrical probe connector cable 52, other types of probes such as visible an invisible light probes can be provided with fiber optic carriers 28 that also transit the length of the interior of the probe housing 24 to terminate at a probe tip opening 27 at the extreme distal end of the probe tip 26. This probe tip opening 27 provides access to the tissue of interest for non-electrical probe types such as, for example, light probes (visible and invisible spectra) 29 and acoustic probes 34 such as ultrasound emitter/receiver probes and passive listening probes, which connect to the sensor module 32 via acoustic conduits 54, as are appropriate for the specific probe type.

As shown in FIG. 2, the operating status of the instrument and the type of tissue identified by the selected probe can be provided to the surgeon visually through the operating indicator lights 64 and the tissue type identification lights 94 on the handle simultaneously with the data transmission to the monitoring workstation 56 and the surgeon worn HUD 60. The HUD 60 can be selectively attached to a faceplate, goggles or other protective or specialized eyewear worn by the surgeon. Preferably, the HUD will not be focused within the normal field of vision for the surgeon as is typical for most HUD applications but instead will be focused slightly above the normal field of vision allowing the surgeon to easily perceive the display of data without interfering with the surgeons normal scan of the surgical site. The HUD 50 may include a small monochrome or color LCD (liquid crystal display) screen displaying relevant information or data. The displayed data may include a combination of colored text, blocks and graphic diagrams representing the type of tissue identified by the selected probe. It is to be understood that the text and graphic may also be monochromatic. Preferably the data displayed for the surgeon through the HUD will be very limited and directed only toward basic requirements so as to not distract the surgeon's attention from the surgery site with information and data that are ancillary to the basic required information of tissue type and/or type of probe selected. In a non-limiting example, the HUD text may simply be letters such as “N”, “A”, “V”, and “O” with each letter indicating Nerve, Artery, Vein, Other tissue respectively. The instrument 10 with the HUD 60 can be capable of programming to adapt to any tissue types within the capability of the sensing probes as desired by the surgeon. A block or graphic can be provided adjacent each tissue type letter displayed in the HUD 60 to provide a redundant visual cue for the surgeon so that the surgeon with an assurance of accuracy viewed and understand the data represented on the HUD 60. Selectively, the text and associated graphics for the identified tissue can be colored and/or animated upon receipt of the data to provide visual confirmation for the surgeon. The HUD 60 can be provided as capable of including high resolutions for displaying more detailed data such as vital statistics of the patient, topography of the anatomical area, internal views during endoscopic surgery, power status, etc.; however, as earlier indicated, it is preferred that the displayed data be that which is minimally required for the operation of the instrument 10 and for the understanding of the type of tissue identified by the instrument 10 so as to minimize distractions imposed upon the surgeon.

An alternative embodiment of a scalpel, generally shown at 200 in FIG. 3, includes a tissue identifying probe, generally shown at 212, integrated with a scalpel blade 220 that is detachable from a handle, which is shown as upper 214A and a lower 214B parts separated for purposes of exposing the interior components. In this alternative embodiment of a scalpel 200, the probe tip 226 with the supporting probe housing 218 is integrated into the detachable scalpel blade 220. Since data must be transferred between the probe 212, and the handle 214, both the detachable blade 220 and the handle 214 are provided with means for establishing an electrical, circuit connection between them. In this example the electrical connection is established by the tang 224 of the blade 220 being inserted into a tang receptacle 225 in the distal end of the handle 214. An electrical interface 228 at that juncture can be provided having a tang receptacle conductive contact 250 on the handle 214 that serves as an electrically conductive connection to a cable contact 252 found at the proximal terminus of the electrical probe connector cable 254. This releasable electrical connection of the tang receptacle conductive contact 250 and the cable contact 252 permits the conduction of an electrical current between the control assembly 244 in the handle 214A-B and the electrical probe 240 located at the probe tip 226. This electrical connection makes possible the transmission of stimulating electrical impulses as well as bounce back impulses from the electrical probe 240 that are conducted to the control assembly 244 and its included processor 246 and transmitter 248. In all other respects, the alternative scalpel 200 functions the same as the first described embodiment of a scalpel shown in FIG. 1. Other suitable types of connector configurations can be alternatively be employed without departing from the spirit of the invention.

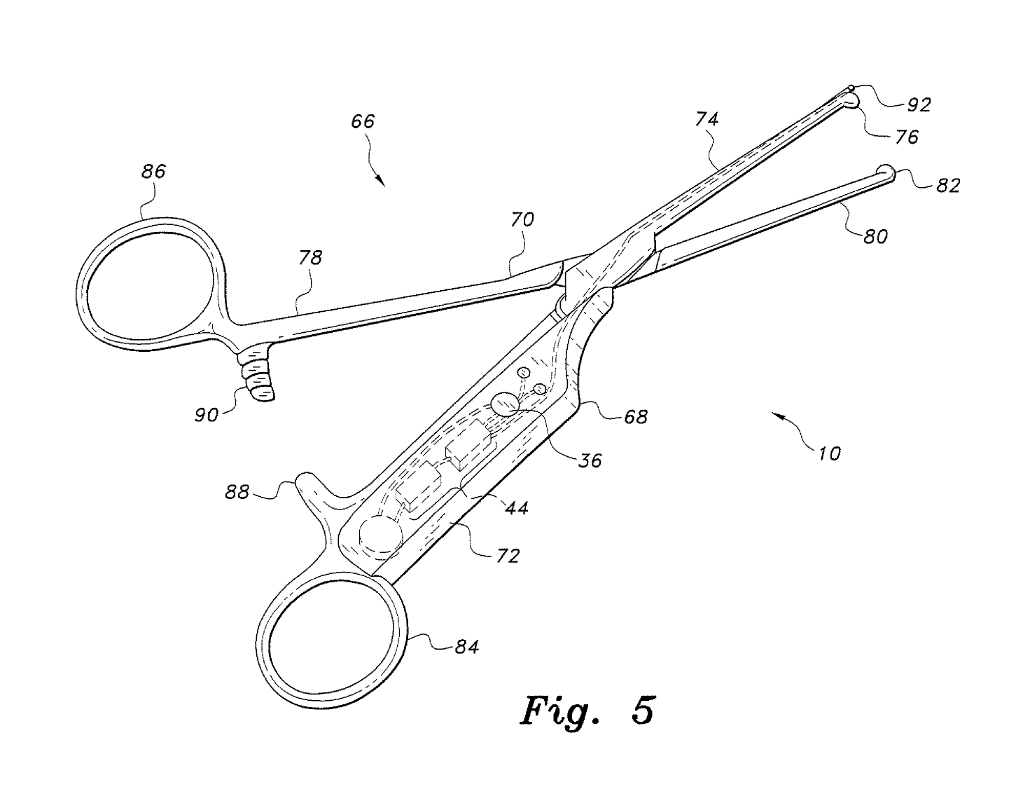

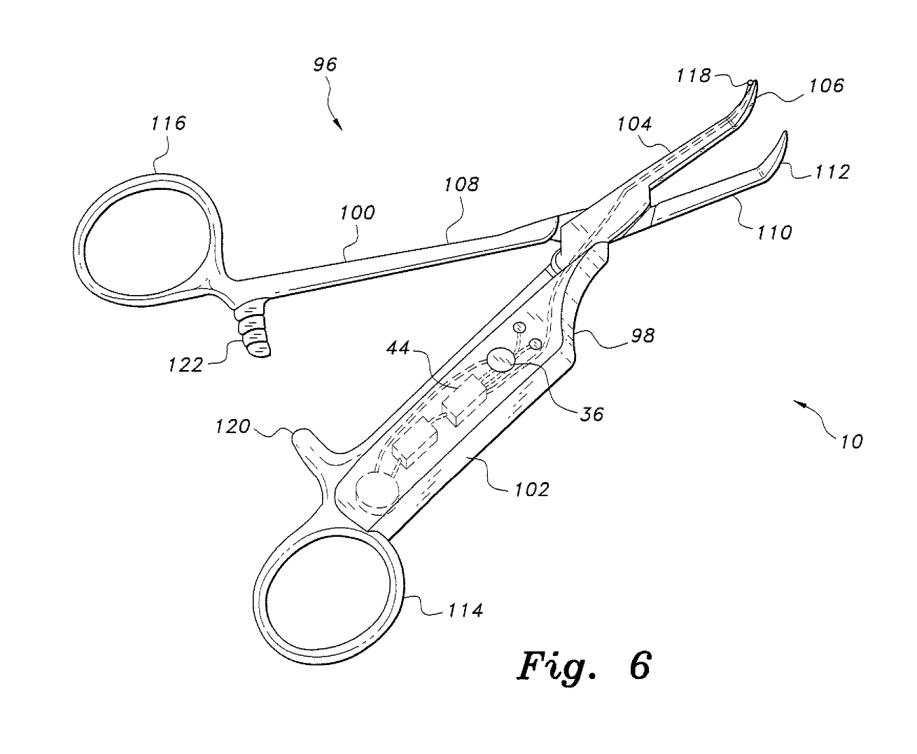

As mentioned above, the tissue-identifying surgical instrument 10 can have multiple embodiments in the form of a variety of surgical instruments, which are used for performing dissections during surgery. Some of the commonly used dissector instruments include forceps 66 and hemostats 96, which are described with reference to FIGS. 5 and 6. FIG. 5 shows a tissue-identifying surgical instrument 10 in the form of forceps 66. The forceps 66 includes an elongate, forceps first arm 68 pivotally attached to an elongate forceps second arm 70. The forceps first arm 68 includes a forceps first handle 72 containing a control assembly 44 therein. A forceps first jaw 74 extends from the forceps first handle 72 terminating with a forceps first hooked tip 76. The forceps second arm 70 is configured substantially similar to the forceps first arm 68. The forceps second arm 70 includes a forceps second handle 78 on one side, and a forceps second jaw 80, which extends from the forceps second handle 78 terminating with a forceps second hooked tip 82. Each forceps handle 72, 78 includes respective forceps finger loops 84, 86 and forceps friction locks 88, 90 as is known in the art.

The forceps 66 is very similar to the conventional forceps as known in the art except for the configuration of the forceps first handle 72 and the forceps probe 92 extending from the forceps first jaw 74. The forceps first handle 72 houses the components for the control assembly 44. For that reason, as shown in FIG. 5, the design of the forceps first handle 72 is enlarged over conventional forceps in order to contain the components of the control assembly 44. The forceps first handle 72 also includes a depressible selector/actuator 36, which is preferably a button having a waterproof coating for selective activation of the forceps probe functions. The forceps 66 with an integrated forceps probe 92 is configured to enable the surgeon to manipulate the forceps 66 and the forceps probe 92 to identify the tissue of interest prior to clamping the tissue using only one hand. The forceps 66 can also be provided as a multiprobe instrument with similar components and capabilities to the multiprobe embodiment of the scalpel shown in FIG. 3.

As shown in FIG. 6, another non-limiting example of the surgical instrument 10 can be provided as a hemostat 96. The combined hemostat instrument 96 with an integral hemostat probe 118 is configured substantially similar to the above forceps 66 with minor differences related to their surgical function. The hemostat 96 includes an elongate, hemostat first arm 98, which is pivotally attached to an elongate, hemostat second arm 100. The hemostat first arm 98 includes a hemostat first handle 102 containing a control assembly 44 therein. A hemostat first jaw 104 extends from the hemostat first handle 102 terminating with a hemostat first hooked tip 106. The hemostat second arm 100 is configured substantially similar to the hemostat first arm 98. The hemostat second arm 100 includes a hemostat second handle 108 on one side, and a hemostat second jaw 110 extends from the hemostat second handle 108 that terminates with a hemostat second hooked tip 112. Each hemostat handle 102, 108 includes respective hemostat finger loops 114, 116 and hemostat friction locks 120, 122 as known in the art.

The hemostat 96 is very similar to the conventional hemostat as known in the art except for the configuration of the hemostat first handle 102 and the hemostat probe 118, which extends from the hemostat first jaw 104. The hemostat first handle 102 houses the components of the control assembly 44. As shown in FIG. 6 the design of the hemostat first handle 102 is enlarged over conventional hemostat in order to contain the components of the control assembly 44. The hemostat first handle 102 also includes a depressible selector/actuator 36, which is preferably an actuator button having a waterproof coating for selective activation of the hemostat probe functions. The hemostat 96 with an integrated hemostat probe 118 is configured to enable the surgeon to manipulate the hemostat 96 and the hemostat probe 118 to identify the tissue of interest prior to clamping the tissue or blood vessel. The hemostat 96 can also be provided as a multiprobe instrument with similar components and capabilities to the multiprobe embodiment of the scalpel shown in FIG. 3.

Thus, it can be seen that the tissue-identifying instrument 10 provides substantial timesaving and safety features. The integrated probe or multiple probes in the surgical instruments provide significant efficiencies and reduce the time and effort required the conventional practice of using a surgical instrument and handling a separate probe. The wireless transmission of the sensed data frees the surgeon from extraneous and potentially hazardous concerns such as the physical interference caused by the location of probe power and data transfer wires. Moreover, the surgeon's concentration on the tissue under examination would be fully engaged with minimal to no potential interruption from operating a separate probe device. The instrument located LED operating lights 64 and the provision of a HUD 60 further enhances the benefits and efficiencies provided by the instrument 10 because the surgeon is able to operate the probe 12 and determine the tissue type without being distracted from the surgical site.

It is to be understood that the tissue-identifying surgical instrument 10 encompasses a variety of surgical instrument embodiments. For example, the tissue identification probe and wireless features are not limited to dissection instruments. These features may be integrated in other surgical instruments such as endoscopes, clamps, scissors, etc. The handle, probe, and the control assembly 44 may be constructed in modular form such that various components may be interchangeable or replaceable with other components depending on the specific sensing function required by the user. Furthermore, the instrument 10 can easily be constructed in modular form to allow the surgical working section of the instrument, e.g., blade or jaws, to be easily changed with different working sections, which would also have the additional tissue-identifying capability.

It is to be understood that the present invention is not limited to the embodiments described above, but encompasses any and all embodiments within the scope of the following claims.

We claim:

1. A tissue-identifying surgical instrument, comprising:
a surgical instrument having at least one handle;
at least one probe connected to the at least one handle, the at least one probe being capable of stimulating a tissue of interest;
a control assembly disposed within the handle, the control assembly having a wireless transmitter for transmitting data to at least one receiving station; and
a power source connected to the control assembly to provide power for operation of the control assembly.

2. The tissue-identifying surgical instrument according to claim 1, wherein said control assembly comprises a sensor module connected to said at least one probe and a processor connected to the sensor module and said wireless transmitter, the sensor module converting data from said at least one probe to be processed by the processor for wireless transmission.

3. The tissue-identifying surgical instrument according to claim 2, wherein said at least one probe is selected from a group consisting of electrical probes, light probes, radio frequency probes, acoustic probes, and combinations thereof.

4. The tissue-identifying surgical instrument according to claim 2, further comprising an actuator disposed on said handle, the actuator being connected to said control assembly and selectively actuated to activate functions of the tissue-identifying instrument.

5. The tissue-identifying instrument according to claim 4, wherein said actuator comprises a button having a protective, insulated and watertight covering.

6. The tissue-identifying instrument according to claim 2, further comprising a plurality of indicator lights disposed on said at least one handle, each of the lights being a different color, the lights being connected to said control assembly, the indicator lights being selectively illuminated in different colors to provide visual confirmation of functions of the tissue-identifying instrument.

7. The tissue-identifying instrument according to claim 6, further comprising a protective, insulated, watertight and translucent covering disposed over said plurality of indicator lights.

8. The tissue-identifying instrument according to claim 1, further comprising a heads-up-display (HUD) operatively connected to said control assembly, the HUD providing localized, visual data to the user on functions of the tissue-identifying instrument.

9. The tissue-identifying instrument according to claim 1, further comprising an insulated, watertight covering surrounding at least said at least one handle.

10. The tissue-identifying instrument according to claim 1, wherein said surgical instrument comprises:
a scalpel, said at least one handle being a scalpel handle;
a scalpel blade attached to one end of the scalpel handle; and
a probe housing attached to the scalpel handle, the probe housing containing said at least one probe and extending above the scalpel blade, the probe housing having an opening, said at least one probe having a probe tip extending through the opening in the probe housing and protruding parallel to the scalpel blade.

11. The tissue-identifying instrument according to claim 10, wherein said scalpel blade comprises a detachable blade having means for selective mounting of the detachable blade to said at least one handle, a back and a tang; said probe housing extending from the back of the detachable blade; the detachable blade having an electrical conduit disposed on the tang to provide an electrical connection between said at least one probe and said control assembly.

12. The tissue-identifying instrument according to claim 1, wherein said surgical instrument comprises forceps having:
an elongate first arm defining a first handle, the first arm having a first jaw extending from one end of the arm, the first jaw terminating at a first hooked tip, the first arm having a first finger loop extending from the opposite end thereof and a first friction lock disposed adjacent the first finger loop;
an elongate second arm pivotally attached to the first arm, the second arm defining a second handle, the second arm having a second jaw extending from one end of the arm, the second jaw terminating at a second hooked tip, the second arm having a second finger loop extending from the opposite end thereof and a second friction lock disposed adjacent the second finger loop; and
a probe housing extending from the forceps first jaw, the probe housing containing said at least one probe and having an opening, said at least one probe having a probe tip extending through the opening in the probe housing adjacent the first jaw.

13. The tissue-identifying instrument according to claim 1, wherein said surgical instrument comprises a hemostat having:
an elongate hemostat first arm defining a first handle, the first arm having a first jaw extending from one end thereof, the first jaw terminating at a first hooked tip, the first arm having a first finger loop extending from the opposite end thereof and a first friction lock disposed adjacent the first finger loop;
an elongate hemostat second arm pivotally attached to the hemostat first arm; the second arm defining a second handle, the second arm having a hemostat second jaw extending from one end thereof, the second jaw terminating at a second hooked tip, the second arm having a second finger loop extending from the opposite end thereof and a second friction lock disposed adjacent the second finger loop; and
a probe housing extending from the hemostat first jaw, the probe housing containing said at least one probe and having an opening, said at least one probe having a probe tip extending through the opening in the probe housing adjacent the first jaw.

14. The tissue-identifying instrument according to claim 1, wherein said power source comprises at least one rechargeable battery.

15. A tissue-identifying instrument, comprising:
an instrument having at least one handle;
at least one probe connected to the handle, the probe being capable of stimulating a tissue of interest;
a control assembly disposed within the handle, the control assembly having a wireless transmitter for transmitting data to at least one receiving station; and
a power source connected to the control assembly to provide power for operation of the control assembly.

16. The tissue-identifying surgical instrument according to claim 15, wherein said control assembly comprises a sensor module connected to said at least one probe and a processor connected to the sensor module and said wireless transmitter, the sensor module converting data from said at least one probe to be processed by the processor for wireless transmission.

17. The tissue-identifying instrument according to claim 16, wherein said at least one probe is selected from a group consisting of electrical probes, light probes, radio frequency probes, acoustic probes, and combinations thereof.

18. The tissue-identifying instrument according to claim 16, further comprising an actuator disposed on said at least one handle, the actuator being connected to said control assembly and selectively actuated to activate functions of the tissue-identifying instrument.

19. The tissue-identifying instrument according to claim 16, further comprising a plurality of indicator lights disposed on said at least one handle, each of the lights being a different color, the lights being connected to said control assembly, the indicator lights being selectively illuminated in different colors to provide visual confirmation of functions of the tissue-identifying instrument.

20. A method of identifying tissue type during a surgical procedure, the method comprising:
providing a tissue-identifying instrument, the instrument having:
at least one handle;
a probe connected to the at least one handle, the probe being capable of stimulating a tissue of interest;
a control assembly disposed within the at least one handle, the control assembly having a wireless transmitter for transmitting data to at least one receiving station; and
a power source connected to the control assembly to provide power for operation of the control assembly,
contacting the probe to a tissue of interest,
activating the probe in order to produce a tissue-identifying response from the tissue of interest, the probe identifying the type of tissue contacted by the probe.
